(19) United States
(12) Patent Application Publication
(10) Pub. No.: US 2011/0263944 A1
(43) Pub. Date: Oct. 27, 2011

(54) LAPAROSCOPIC RETRACTOR

(76) Inventor: Jerome Cau, Poitiers (FR)

(21) Appl. No.: 12/937,653

(22) PCT Filed: Nov. 7, 2007
(86) PCT No.: PCT/FR07/01838

§ 371 Date: May 23, 2011

(30) Foreign Application Priority Data
Nov. 7, 2006 (FR) 0609723

(51) Int. Cl. A61B 1/32

(52) U.S. Cl. 600/204

(57) ABSTRACT

A laparoscopic spacer (1) to keep a first organ at a distance from a second organ during an operation on said second organ, comprising: a maneuvering grip (2); a tube having as extensions at least two preformed flexible blades (6a, 6b) so that the blades (6a, 6b), in a first position, extend substantially parallel to one another; a smooth net or a smooth membrane extending between the blades (6a, 6b); a sheath (7) arranged to receive the blades (6a, 6b) in their first position and to allow their passage to a second position in which they automatically expand on both sides of said tube. The spacer comprises a return device provided to maintain the gap between the two blades (6a, 6b). The invention also relates to a laparoscopic device comprising such a spacer.

The invention relates to a laparoscopic retractor for keeping a first organ at a distance from a second organ during a laparoscopic surgical intervention.

Such interventions require the use of optical trocars and of surgical trocars through which various surgical instruments are introduced into the peritoneal cavity, which has been insufflated in advance at a constant pressure.

One of the main difficulties during these interventions entails keeping the various organs in a stable position that not only facilitates the work of the surgeon, by suitably exposing the organs to be manipulated, but also avoids any distress, in particular respiratory distress, in the patient during the intervention.

The position of the intestinal loops is a particular concern insofar as they are relatively voluminous and mobile. For example, in interventions of the type involving restoration of the abdominal aorta, the intestinal loops prevent easy access to the aorta and form an obstacle to the surgeon's field of view.

Various approaches have been proposed for getting round this difficulty. One of these approaches is to use a device of the retractor type with which the visceral organs that may get in the way of the operation can be held aside and maintained in position.

Document WO 06/069947 describes a retractor-retainer of intestines for celioscopic surgery which comprises several elements that have to be inserted into one another by the surgeon during the intervention in order to adapt to the anatomy of the patient. Such a device is complicated to maneuver for the surgeon. In addition, this retractor cannot be used for fully laparoscopic interventions in which the entire procedure is performed by celioscopy, including the stage of surgical restoration. It is in fact intended for minimally invasive surgical procedures or for combined procedures in which, after a first stage of dissection under celioscopy, the actual restoration itself is performed by way of an abdominal incision without celioscopy, as in conventional surgery.

U.S. Pat. No. 5,178,133 also discloses a laparoscopic retractor comprising two arms between which there extends an elastic membrane designed to retain the organs or tissues in question, the two arms being able to be opened or closed like scissors in order to adjust the distance between them. This retractor has the disadvantage of not being able to be used for complex restoration procedures, for example on the aorta which is situated deep in the abdomen, against the thoraco-lumbar spine, and is consequently difficult to access. The use of this retractor is therefore limited to the maneuvering of intra-abdominal organs that are directly accessible, for example the liver. In addition, because of its rigidity, a retractor of this kind may cause damage to the solid organs during its manipulation within the abdominal cavity. Another retractor of this type is described in application no. WO 95/07052 and comprises two rigid blades which carry a flexible net and which are able to pivot and move away from each other at an angle that is controlled by a rigid wire connecting the base of the blades to a slide, which can be maneuvered from the direction of the handle of the retractor. Application no. WO 03/094744 describes a retractor comprising a shaft acting on several rigid blades which can move by pivoting and are interconnected by a net.

Document U.S. Pat. No. 5,755,661 describes a retractor comprising a plurality of arms that are able to deploy so as to form a plane surface. This retractor is designed more especially to enlarge the abdominal wall during an intervention and does not therefore permit retraction of a specific organ in order to facilitate access to the organ or to the tissue that is to be manipulated by the surgeon.

Consequently, the retractors known from the prior art are not entirely satisfactory since, in some cases, they are complicated to manipulate, which makes precise control of the surgical procedure more difficult for the surgeon to acquire and entails longer operating times. In addition, some of them cannot be used in procedures performed entirely by laparoscopy, and they may also cause damage to the organs surrounding them.

The publication by J. Cau et al., Journal of Vascular Surgery, vol. 41, no. 5 (2005), pages 902-906, concerns a retractor comprising two blades which are arranged in a sheath and between which a retention net is provided for enveloping and retaining the intestinal loops. The blades are able to open when they are pushed out of the sheath, which leads to the deployment of the net in the abdominal cavity.

The invention proposes a retractor designed to permit automatic deployment of the blades and precise adjustment of the distance between them. This retractor has the advantage of being easy to manipulate, being able to be used in various types of interventions, being easily adaptable to the morphology of each patient, and of permitting precise and rapid maneuvers by the user.

To this end, the subject matter of the invention is a laparoscopic retractor for keeping a first organ at a distance from a second organ during a laparoscopic surgical procedure performed on said second organ, said retractor comprising:
a maneuvering handle that can be fixed to an operating table;
a hollow rigid tube having a proximal end connected to the handle and a distal end continued by at least two preformed flexible blades, one end of each blade being fixed to the distal end of the tube in such a way that the blades, in a first position, extend substantially parallel to each other;
a flexible net or a flexible membrane extending between the blades, said net or said membrane having sufficient dimensions to at least partially envelop said first organ;
a substantially cylindrical sheath designed to receive the blades in their first position, said sheath being mounted so as to slide on said tube in such a way that, during its sliding movement toward the tube, it allows the blades to change from their first position to a second position in which they automatically deploy on both sides of said tube.

Said retractor comprises a return device designed to adjust and maintain the spacing between the blades, said device having return elements composed of at least one flexible plate which has two branches forming substantially a V and which is arranged between said blades, each end of the branches of the V being connected respectively to each of the two blades, or of a check-screw or drilled ball, in proximity to the distal end of said tube, and control means for controlling the return elements, said control means being arranged on the handle.

Such a device has the advantage of deploying automatically when placed at the desired location, so as to constitute a barrier and prevent the visceral organs from invading the operating field. It thus ensures that the organ to be operated on is exposed in a stable manner, without any other manipulation, and without the need for an abdominal incision. In addition, the return device allows the spacing between the blades to be adapted easily and precisely to the dimensions of the organ that is to be retained.

The fact that the retractor according to the invention is able to deploy automatically, and can be adjusted precisely, also limits the manipulations that have to be performed by the surgeon, which reduces the risk of damage to the surrounding organs or tissues. This feature allows the retractor according to the invention to be used according to the NOTES (Natural Orifice Transluminal Endoscopic Surgery) technique. Thus, the surgical intervention is performed without any cutaneous incision, except for introduction of a 2-mm needle for insufflation and control of the intra-abdominal pressure, the instruments being introduced in particular either by the transgastric route or by the transvaginal or transrectal route. This surgical technique has the advantage of reducing or even eliminating postoperative pain, of making access to certain organs easier, and of avoiding trauma to the abdominal wall. It also has a cosmetic advantage in the sense that it avoids scarring.

According to one embodiment, the return elements are composed of at least one flexible plate with two branches forming substantially a V, each end of the branches of the V being connected respectively to each of the two blades, and a threaded rod penetrating at least partially into said tube, said threaded rod having a distal end that can extend outside the tube, when the blades are in their second position, and that comes into abutment against the at least one plate in the V formed by the two branches. According to this embodiment, the control means are composed of a knurled wheel cooperating with the threaded rod.

Alternatively, provision can be made for the return elements to be composed of a check-screw of conical shape or inverted pyramid shape, or of a drilled rod, and for the control means to be composed of a system comprising a nut and a counter-nut.

According to one embodiment, the distal end of each branch of the V-shaped plate slides in a channel formed in the inner face of the blade of the retractor, the effect of which is to maintain the return element in an optimum position, in contact with the inner face of the branch of the retractor, and of making the sliding movement easier.

The preformed blades are preferably made of a flexible metal of the steel type or of an injected plastic material.

When a net is used between the blades, the net is in the form of a polypropylene mesh, for example.

The automatic and atraumatic spacing of the blades of the retractor is made possible by the particular shape of the blades and of the net.

For this purpose, each of the blades has an S-shape when they are free and are not connected by the net. The frame of the net is formed by the two separate blades attached to the distal end of the sleeve via their proximal end.

The angle formed by the sleeve and the straight line connecting the distal and proximal ends of each blade, before placement of the net, is preferably between 90° and 110°.

Moreover, the initial S-shape of each blade means that the net can be given a concave shape when deployed, making the net more effective for holding an organ such as the small intestine. This shape also has the advantage of being more anatomical and causing less trauma.

When the net is placed on the blades, its configuration keeps each blade in an angled position of about 30° with respect to the axis of the handle. This angle difference between the positions of the blades before and after placement of the net is the reason for the smooth and atraumatic deployment of the retractor.

The deployment force of the net allows it to expand to its final shape without any other external manipulation, and without it being necessary to use other instruments introduced into the abdominal cavity via trocars, which is the case in the known retractors and which requires multiple incisions. The retractor according to the invention thus overcomes this disadvantage.

Moreover, in order to deploy, the retractor according to the invention does not require an adjusting mechanism, such as a screw mounted so as to rotate relative to the handle, and this reduces the risk of organs being injured.

The return device allows the retractor to be easily strengthened once it has been deployed and positioned.

According to one variant, the two blades can be interconnected so as to form a single blade fixed to the sleeve of the retractor.

The invention also relates to a laparoscopic device comprising a retractor as defined above and an endoscopic trocar, said retractor being placed inside said trocar.

The laparoscopic retractor according to the invention can be supplied to the surgeon with the net in place on the blades, or with the net not fitted, that is to say leaving the blades free, in which case the surgeon has to fit the net on the blades at the time of the intervention.

Other features and advantages of the invention will become clear from the following description and by reference to the attached drawings, in which:

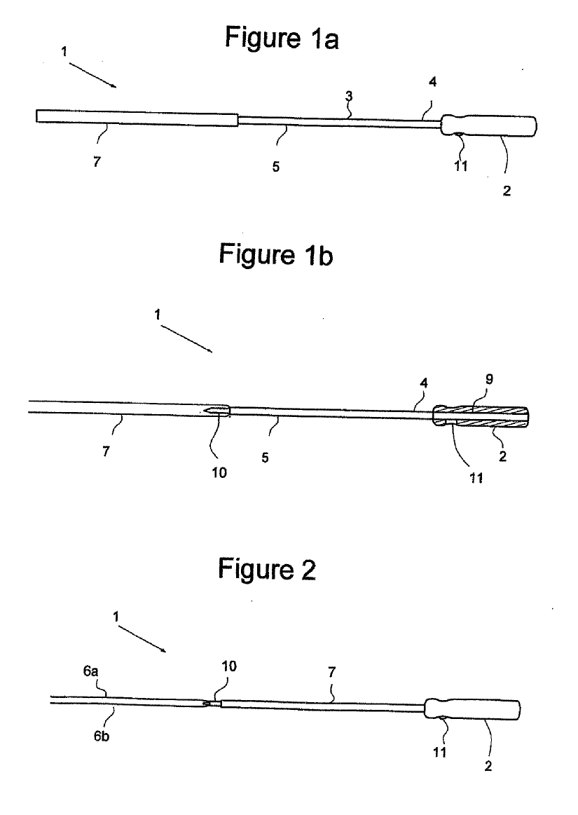

FIGS. 1a and 1b are a perspective view and a longitudinal sectional view, respectively, of a retractor according to the invention, in which the blades are arranged inside the sheath, in their first position;

FIG. 2 is a perspective view of a retractor according to the invention in which the sheath has slid on the rigid tube, the blades being in the process of changing to their second position;

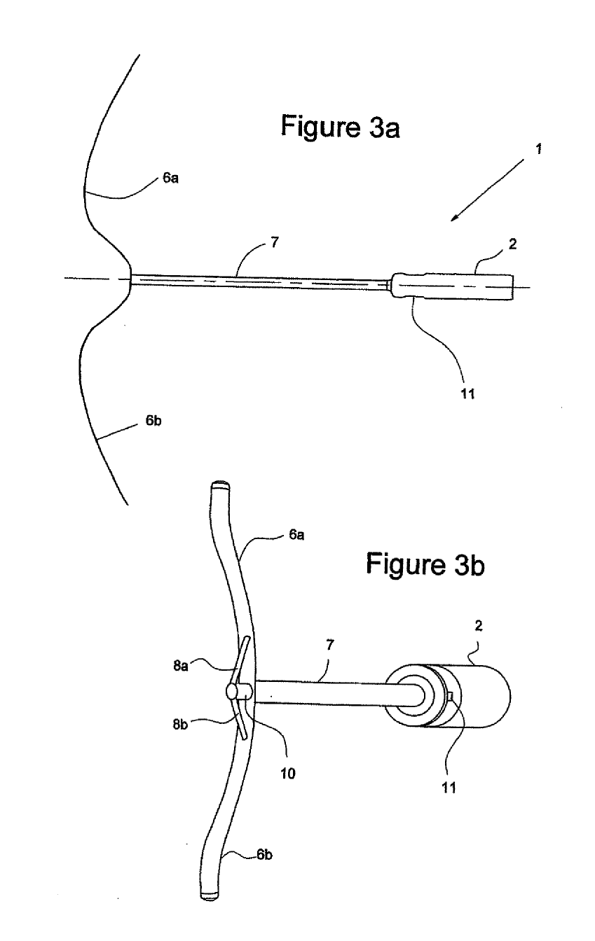

FIGS. 3a and 3b are a side view and a rear view, respectively, of a retractor that has no net or membrane, showing the maximum spacing that the blades could reach upon their automatic deployment;

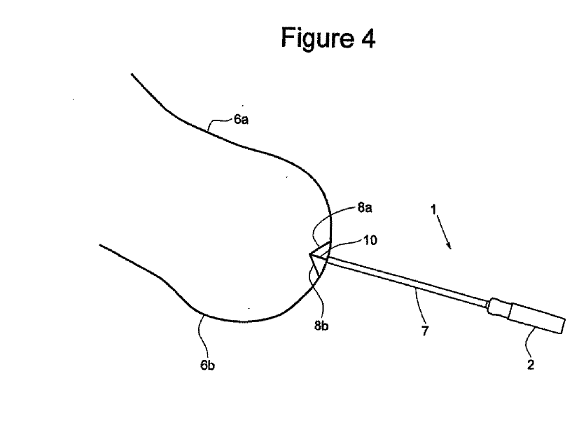

FIG. 4 shows a perspective view of a retractor according to the invention in which the blades are in their second position, the spacing between the blades having been adjusted with the aid of the return device.

The retractor 1 shown in the figures is designed more particularly for retracting the intestinal loops during a laparoscopic intervention involving restoration of the abdominal aorta.

For this use, the retractor 1 is placed inside an endoscopic trocar (not shown) whose diameter is approximately equal to 10 mm.

The retractor 1 comprises a substantially cylindrical handle 2 provided with means (not shown) for fixing it to an operating table.

The handle 2 is continued by a rigid hollow tube 3 whose proximal end 4 is connected to the handle 2 and whose distal end 5 is continued by two flexible blades 6a, 6b.

The terms “proximal” and “distal” designate the parts of the device that are situated toward the user and away from the user, respectively.

Before being mounted on the retractor 1, the two blades 6a, 6b have been preformed substantially in the shape of an S, such that they are subsequently able to deploy into a precise configuration.

One of the ends of each blade 6a, 6b is fixed to the distal end 5 of the rigid tube 3, such that the blades 6a, 6b, in a first position, are substantially parallel to each other (see FIG. 1b).

The retractor 1 also comprises a flexible net (not shown) arranged between the two blades 6a, 6b and fixed to these via its edges.

This net has sufficient dimensions to at least partially envelop the abdominal loops. Its dimensions are adapted to the size or volume of the patient's abdominal cavity. For a small cavity with a volume of less than 3 liters after insufflation of the pneumo-peritoneum, the dimensions of the net are 10 cm in width and 15 cm in length when deployed, the width corresponding to the dimension of the net between the two deployed blades. For a cavity considered as normal, and with a volume of between 3 and 4.5 liters after insufflation of the pneumoperitoneum, the dimensions of the net are 15 cm in width and 20 cm in length when deployed, and, for a very large abdominal cavity with a volume greater than 4.5 liters after insufflation of the pneumoperitoneum, the dimensions of the net are 15 cm in width and 25 cm in length when deployed. The dimensions of the blades are adapted to those of the net, their thickness being about 1 mm and their width about 3 mm.

A substantially cylindrical sheath 7 is provided on the retractor 1 in order to receive the blades 6a, 6b in their first position (FIGS. 1a and 1b). Inside the sheath 7, the blades 6a, 6b are in contact with each other by way of their convex edges.

The dimensions of the sheath 7 are designed to allow it not only to fully receive the blades 6a, 6b in a substantially parallel position, but also to be able to slide on the rigid tube 3, as is shown in FIG. 2, in such a way as to release the blades 6a, 6b and allow them to change to a second position.

The retractor 1 is also provided with a return device that is able to adjust and maintain the spacing between the blades 6a, 6b when they are placed in their second position by deploying automatically.

According to the embodiment shown in the figures, the return device comprises return elements formed by a flexible plate with two branches 8a, 8b that are arranged between the blades 6a and 6b (see FIGS. 3b and 4). The two branches 8a, 8b together form a V, each end of which is connected to one of the two blades 6a, 6b. The ends of the two branches 8a and 8b of the flexible plate bear on the inner face of the blades 6a and 6b on which they are able to slide, and their movement is controlled by the edges of the blades 6a and 6b that are raised to ensure that the ends of the branches 8a and 8b are kept in place. Thus, the blades have a U-shaped cross section, the wings of the U being formed by the edge of the blades. The return elements also comprise a threaded rod 9 extending from the handle 2 of the retractor 1 to the flexible blades 6a, 6b and penetrating at least partially into the rigid tube 3. The distal end 10 of the threaded rod 9 extends outside the rigid tube 3 and comes into abutment against the plate, in the V formed by the two flexible branches 8a and 8b.

The return device also comprises control means which permit control of the return elements and are composed of a knurled wheel 11 arranged on the handle 2 and designed to cooperate with the threaded rod 9.

The function of the illustrated retractor during a laparoscopic intervention for restoration of the abdominal aorta will now be described.

Before the retractor 1 is introduced into the abdominal cavity, the operating table must be tilted 30 degrees to the right to ensure that the intestinal loops of the patient are located in the right-hand part of the abdomen. The retractor 1 is then introduced into the iliac fossa by way of the trocar. The two blades 6a, 6b are then in their first position, inside the sheath 7.

The two blades 6a, 6b are then pushed out of the sheath 7 by means of a simple rearward movement of the sheath, said sheath 7 being able to slide on the rigid tube 3. The blades 6a, 6b are thus able to deploy automatically. FIGS. 3a and 3b show the theoretical maximum spacing that the blades could reach, thus adopting their S-shaped configuration, if there were no net or membrane provided between them. In practice, when the blades deploy automatically, the presence of the net, which also deploys automatically, limits the spacing between the blades.

The surgeon is then able to adjust the spacing between the blades 6a, 6b by actuating the knurled wheel 11 in such a way as to place the blades 6a, 6b in their second position.

If the knurled wheel 11 is turned in order to move the threaded rod 9 out of the tube 3, the rod will come to bear against the plate, in the V formed by the branches 8a, 8b, and will reduce the angle between the two branches 8a, 8b. Such a movement has the effect of narrowing the space between the two blades 6a, 6b by moving these toward each other (FIG. 4).

If, by contrast, the knurled wheel 11 is turned in such a way as to move the threaded rod 9 inside the tube 3, the angle between the two branches 8a, 8b will be increased under the effect of the elasticity of the plate, and the two plates 6a, 6b will move slightly further away from each other. Such a movement is in practice intended to exert a force for maintaining a steady distance between the blades 6a, 6b. This can be particularly useful when the blades have been confined for a long time in the sheath, the result of which is a less stable configuration of the blades 6a, 6b upon their automatic deployment.

The adjustment made by the surgeon makes it possible to deploy the net and give it the shape and size suitable to envelop and retain the intestinal loops during the intervention.

When the retractor 1 is correctly positioned in the abdomen, around the intestinal loops, the operating table can be reset and the patient is then placed lying on his back.

The intervention on the abdominal aorta is performed, after which the retractor 1 is simply withdrawn by bringing the blades 6a, 6b back inside the sheath 7, which also causes the net to fold up automatically without any further maneuvering.

Depending on the materials used to produce it, the retractor 1 according to the invention can be reused or can be disposed of.

Alternatively, the net can be disposable and the handle and blades of the retractor can be reused.

1. A laparoscopic retractor for keeping a first organ at a distance from a second organ during a laparoscopic surgical procedure performed on said second organ, said retractor comprising:
a maneuvering handle that can be fixed to an operating table;
a hollow rigid tube having a proximal end connected to the handle and a distal end continued by at least two preformed flexible blades, one end of each blade being fixed to the distal end of the tube in such a way that the blades, in a first position, extend substantially parallel to each other;
a flexible net or a flexible membrane extending between the blades, said net or said membrane having sufficient dimensions to at least partially envelop said first organ;
a substantially cylindrical sheath designed to receive the blades in their first position, said sheath being mounted so as to slide on said tube in such a way that, during its sliding movement toward the tube, it allows the blades to change from their first position to a second position in which they automatically deploy on both sides of said tube;
wherein said retractor has a return device designed to adjust and maintain the spacing between the blades, said device having return elements composed of at least one flexible plate which has two branches forming substantially a V and which is arranged between said blades, each end of the branches of the V being connected respectively to each of the two blades, or of a check screw or a drilled ball, in proximity to the distal end of said tube, and control means for controlling the return elements, said control means being arranged on the handle.

2. The retractor as claimed in claim 1, wherein the return elements are composed of at least one flexible plate with two branches forming substantially a V, each end of the branches of the V being connected respectively to each of the two blades, and of a threaded rod penetrating at least partially into said tube, said threaded rod having a distal end that can extend outside the tube when the blades are in their second position, and that comes into abutment against the at least one plate in the V formed by the two branches, and in that the control means are composed of a knurled wheel that cooperates with said threaded rod.

3. The retractor as claimed in claim 1, wherein the return elements are composed of a check-screw or a drilled ball, and in that the control means are composed of a system comprising a nut and a counter-nut.

4. The retractor as claimed in claim 1, wherein the distal end of each branch of the V-shaped plate slides in a channel formed in the inner face of the blade of the retractor.

5. The retractor as claimed in claim 1, wherein the blades are made of a flexible metal of the steel type or of an injected plastic material.

6. The retractor as claimed in claim 1, wherein the net is made of polypropylene.

7. A laparoscopic device comprising a retractor as claimed in claim 1 and an endoscopic trocar, wherein said retractor is placed inside said trocar.
